(19) United States
(12) Patent Application Publication
(10) Pub. No.: US 2013/0109968 A1
(43) Pub. Date: May 2, 2013

(54) ULTRASONIC IMAGING DEVICE

(71) Applicant: Takashi Azuma, Fuchu (JP)

(72) Inventor: Takashi Azuma, Fuchu (JP)

(73) Assignee: HITACHI MEDICAL CORPORATION, Tokyo (JP)

(21) Appl. No.: 13/808,255

(22) PCT Filed: Jul. 15, 2011
(86) PCT No.: PCT/JP2011/066220

§ 371 Date: Jan. 4, 2013

(30) Foreign Application Priority Data
Jul. 15, 2010 (JP) 2010-160205

(51) Int. Cl. A61B 8/14; A61B 8/08; A61B 8/06; A61B 8/00

(52) U.S. Cl. CPC A61B 8/145; A61B 8/461; A61B 8/54; A61B 8/5269; A61B 8/488; A61B 8/06
USPC 600/441; 600/447

(57) ABSTRACT

Disclosed is an ultrasonic imaging device having an improved signal-to-noise ratio. After A/D conversion, by performing filtering between channels or two-dimensional filtering between channels and along the time axis, reduction in spatial resolution is suppressed as much as possible to thereby improve the signal-to-noise ratio.

### TECHNICAL FIELD

The present invention relates to a medical ultrasonic imaging device, and particularly to a technique of high image quality by improving a signal to noise ratio.

### BACKGROUND ART

An ultrasonic diagnosing device has been widely used as a medical cross-sectional image imaging device due to characteristics such as real-time properties and portability. The ultrasonic diagnosing device has been absolutely essential as a medical image diagnosing device in a method of detecting, especially, lesions of tumors of soft tissue such as body surface tissue such as the mammary gland or thyroid gland, digestive organs such as the liver or kidney, and circulatory organs such as the heart or vascular channels. A spatial resolution and an imaging depth that are fundamental indexes of performance in the ultrasonic diagnosing device have a trade-off relation. Specifically, if the frequency is raised, the wavelength is shortened. If the fractional bandwidth (=center frequency/bandwidth) is constant, the spatial resolution is improved. On the other hand, the attenuation rate along with ultrasonic propagation in a living body is increased as the frequency becomes higher. Namely, the higher the frequency is, the more the signal strength at a position far from an ultrasound probe is extremely attenuated. Ultrasonic energy attenuated in a living body is changed to heat, and thus there is a possible risk that the living body is damaged by applying an excessive acoustic pressure. Further, if a temporal peak pressure becomes large, the risk of cavitation is possibly increased. In order to minimize the risk of damage of the living body, the acoustic pressure or energy of ultrasonic waves transmittable to the living body is restricted. Thus, in the case where echoes from a deep area are reduced as a result of tissue attenuation, there is no method to restore. In a normal diagnosing device, after an echo signal is received and amplified by a preamplifier, analog to digital conversion is performed. Thus, every diagnosing device has a finite dynamic range due to the finite bit width of analog to digital conversion. Further, under the influence of random electric noise after analog to digital conversion, the fact is that the dynamic range of analog to digital conversion cannot be fully used in general. Therefore, in the case where echoes from a tissue deep area are reduced due to tissue attenuation, the signal to noise ratio is decreased, resulting in deterioration in the sensitivity and resolution of an image. If the frequency is lowered to compensate this, the spatial resolution is disadvantageously decreased. As a method of compensating such decrease in the signal to noise ratio, there is a method as disclosed in Patent Literature 1 in which after analog to digital conversion and delay processes, a bandpass filter on a frequency space is inserted to improve the signal to noise ratio.

### CITATION LIST

### Patent Literature

Patent Literature 1: Japanese Patent Application Laid-Open Publication No. H11-244286

### SUMMARY OF INVENTION

### Technical Problem

A bandpass filter on a frequency axis is one method contributing to compatibility between a spatial resolution and an imaging depth (penetration). However, in order to significantly improve the signal to noise ratio with this method, it is necessary to narrow the bandwidth of the bandpass filter and there is a possibility to lead to a decrease in the spatial resolution. Namely, this means being not free from the trade-off between the spatial resolution and the imaging depth. An object of the present invention is to realize compatibility between the spatial resolution and the imaging depth of an ultrasonic diagnosing device using other means.

### Solution to Problem

In order to achieve the above-described object, the present invention provides an ultrasonic diagnosing device including: an array element; a transmitter that transmits ultrasonic waves to a target through the array element; a receiver that delays and sums a reception wave signal received by the array element from the target; a display that displays a cross-sectional image of the target from the reception wave signal output from the receiver; and a controller that controls the transmitter and the receiver, wherein the receiver includes an inter-channel filter that selectively suppresses electric noise, a receiving beamformer that selectively receives a beam, and an envelope detection unit, and the inter-channel filter performs a filtering process in which a signal and noise contained in an echo signal from the target are separated from each other using continuity between channels to remove the noise.

Specifically, according to the present invention, filtering between channels or two-dimensional filtering between channels and in a temporal axis is performed after analog to digital conversion, so that a signal to noise ratio can be improved while minimizing a decrease in the spatial resolution.

### Advantageous Effects of Invention

According to the aspects of the present invention, compatibility between a spatial resolution and a signal to noise ratio that are two fundamental performances of an ultrasonic diagnosing device is achieved, and an improvement in the quality of a cross-sectional image is realized as a result.

### BRIEF DESCRIPTION OF DRAWINGS

FIG. 1 is a block diagram for showing a device configuration of an ultrasonic imaging device according to an embodiment of the present invention.

FIG. 2 is a block diagram for showing a device configuration of an ultrasonic imaging device according to a conventional embodiment.

FIG. 3 is an explanatory diagram of the concept of the present invention.

FIG. 4 show results obtained by calculating a strength ratio of a signal to noise in the presence or absence of electric noise and a strength ratio of a signal to noise in the case where a conventional method was applied.

FIG. 5 show results obtained by calculating a strength ratio of a signal to noise with a method of the present invention.

FIG. 6 are explanatory diagrams of a simulation model for studying the present invention.

FIG. 7 are explanatory diagrams related to characteristics of electric noise and an acoustic signal.

FIG. 8 is a block diagram for showing a part of a device configuration of an ultrasonic imaging device according to an embodiment of the present invention.

FIG. 9 are explanatory diagrams of a filtering algorithm of the present invention.

FIG. 10 is an explanatory diagram of a signal processing algorithm of the present invention.

FIG. 11 is a configuration diagram of a device in the case where the present invention is applied to a Doppler blood flow image.

FIG. 12 are explanatory diagrams of the concept of a Doppler signal process.

FIG. 13 is an explanatory diagram of noise targeted by the present invention in the Doppler signal process.

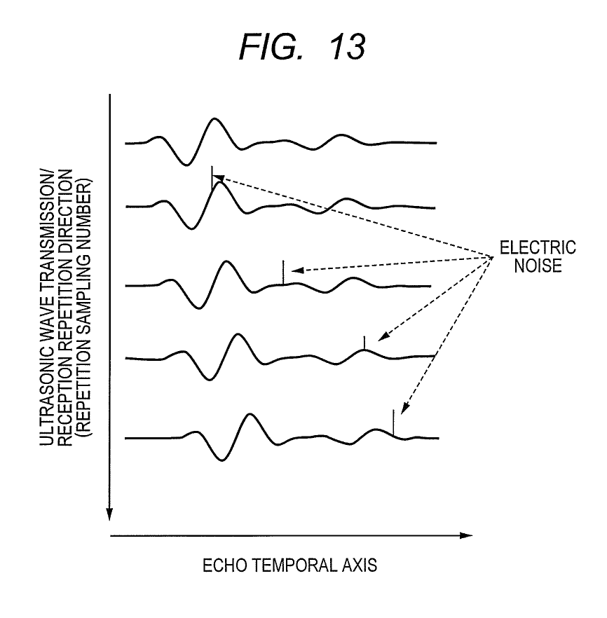

### DESCRIPTION OF EMBODIMENTS

Hereinafter, modes for carrying out the present invention will be described based on the drawings.

### First Embodiment

An ultrasonic diagnosing device according to a first embodiment includes an array element 1, a transmitter that transmits ultrasonic waves to a target through the array element, a receiver that delays and sums a reception wave signal received by the array element from the target, a display 14 that displays a cross-sectional image of the target from the reception wave signal output from the receiver, and a controller 6 that controls the transmitter and the receiver. The receiver includes an inter-channel filter 9 that selectively suppresses electric noise, a receiving beamformer 10 that selectively receives a beam, and an envelope detection unit 11. The inter-channel filter 9 performs a filtering process in which a signal and noise contained in an echo signal from the target are separated from each other using continuity between channels to remove the noise.

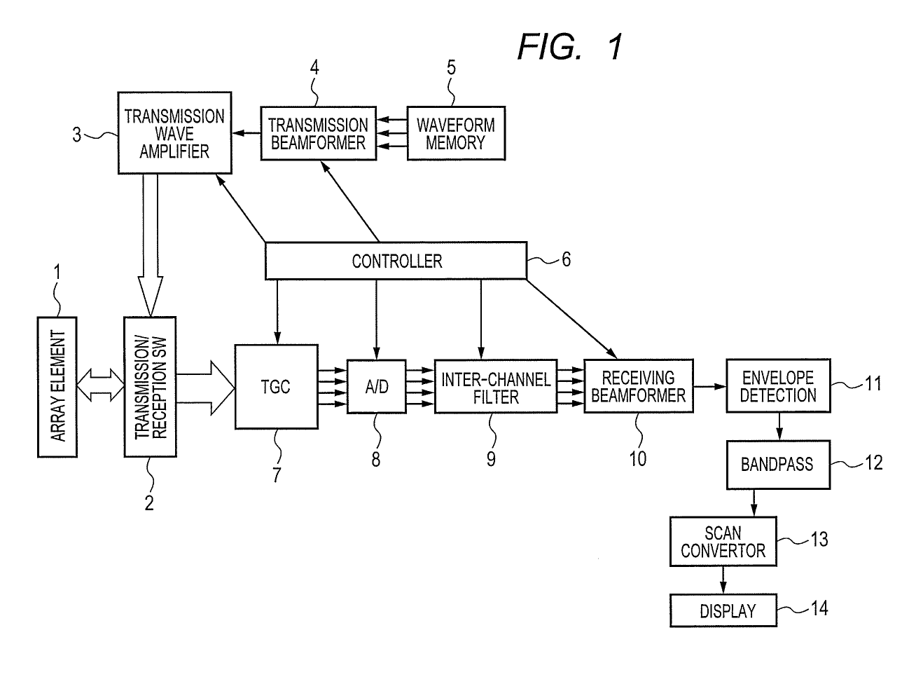

In the first place, a flow of signal processing for imaging in the ultrasonic diagnosing device of the embodiment will be described using FIG. 1. A transmission to waveform stored in a waveform memory 5 is transmitted as an electric pulse to the array element 1 placed on the surface of the target (not shown) through a transmission/reception switch (SW) 2 from a transmission beamformer (BF) 4 through a transmission wave amplifier 3 under the control of the controller 6. The transmission wave amplifier 3, the transmission beamformer 4, and the waveform memory 5 configure the transmitter. In this case, the transmission beamformer 4 controls to optimize delay times between channels (hereinafter, abbreviated as ch in some cases) of the array element 1 so that an ultrasonic beam travels on a desired scan line. In response to an electric signal from the transmission beamformer 4, the electric signal is converted into an ultrasonic signal in the array element 1, and an ultrasonic pulse is transmitted into the target.

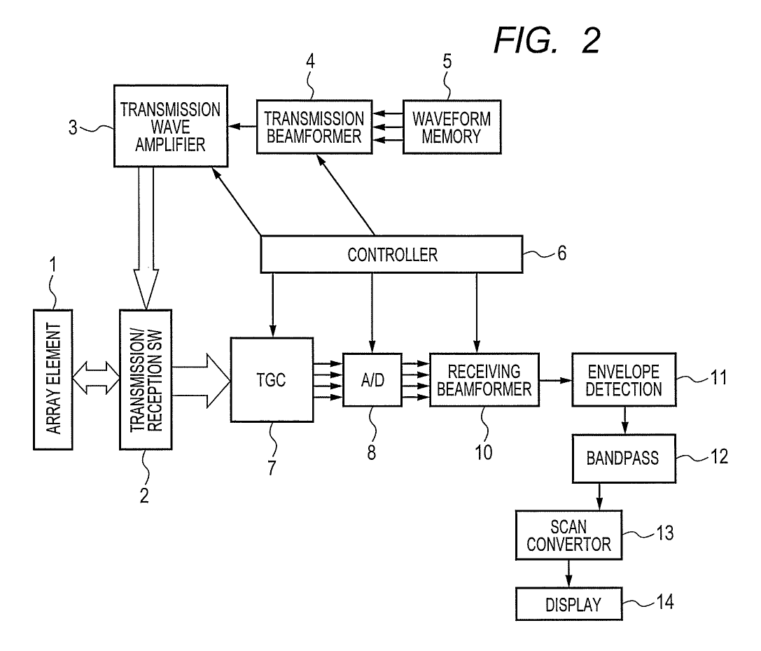

A part of the ultrasonic pulse scattered in the target is received by the array element 1 again as an echo signal to be converted from the ultrasonic signal into an electric signal. The received signal is drawn in the receiver through the transmission/reception switch 2. In the receiver configured using circuit blocks to a bandpass filter 12 from a TGC amplifier 7 into which the received electric signal from the transmission/reception switch 2 is input, the signal is first amplified by the time gain control (TGC) amplifier 7 in accordance with the propagation distance of echoes to convert an analog signal to a digital signal with an analog/digital (A/D) conversion element 8, and a signal to noise ratio is improved by the inter-channel filter 9 as a characteristic of the present invention. In addition, a delay and sum process is performed by the receiving beamformer (BF) 10 for the signal as data on a scan line obtained by selectively intensifying an echo signal from a desired depth on a desired scan line. The data for which the delay and sum process was performed is converted into an envelope signal by the envelope detection unit 11 to be transmitted to a scan convertor 13 through the bandpass filter 12, and scan conversion is performed. Data obtained after the scan conversion is transmitted to the display 14, and is displayed as an ultrasonic cross-sectional image. The receiver of the ultrasonic diagnosing device in the embodiment includes at least the inter-channel filter 9 that selectively suppresses electric noise, the receiving beamformer 10 that selectively receives a beam, and the envelope detection unit 11. For reference, an example of a configuration of a conventional device is shown in FIG. 2. The configuration of the conventional device is the same as that of the present invention except that no inter-channel filter 9 is provided after the analog to digital conversion element 8.

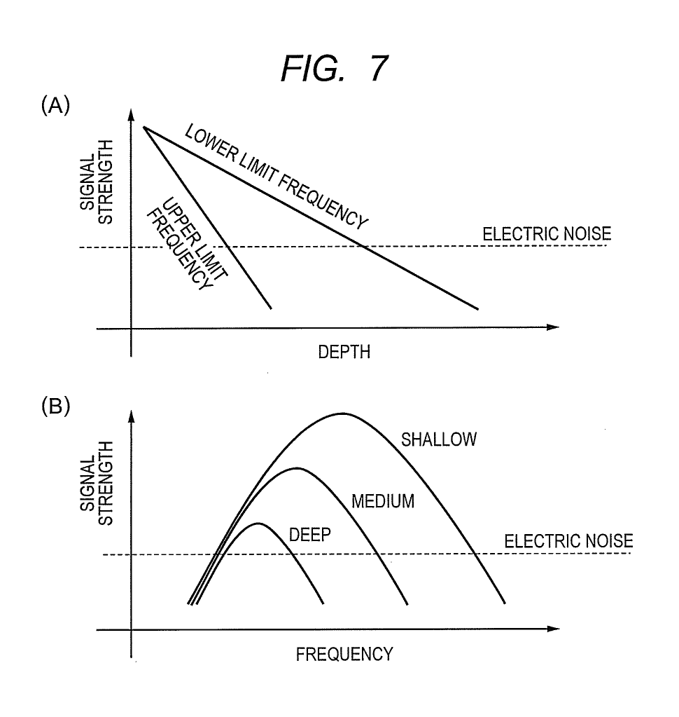

Next, the concept of the present invention will be described. An object to be separated and removed in the present invention is electric noise that is mixed in the course from the time an ultrasonic signal is converted into an electric signal by the array element 1 to the time the delay and sum process is performed by the receiving beamformer 10. FIG. 7 are outline diagrams each showing electric noise and acoustic signal characteristics at each frequency. In each diagram, the vertical axis represents the strength (each component for the signal) of signal noise. The horizontal axis represents a depth (propagation distance of the signal) in FIG. 7(a), and the horizontal axis represents a frequency in FIG. 7(b). The noise is substantially flat without being dependent on the depth (corresponding to the reception times of echoes) and the frequency. On the other hand, the signal strength is decreased in accordance with the depth. The higher the frequency is, the greater the inclination is. The lower the frequency is, the smaller the attenuation is. However, the spatial resolution is also deteriorated. Thus, the frequency at which a necessary signal to noise ratio can be obtained at the deepest position of the imaging depth becomes the lower limit frequency in the imaging conditions, and differs depending on a measurement region or a disease. In addition, the frequency is adjusted in accordance with the preference of a user in some cases. If a high frequency is used, a high degree of spatial resolution can be realized. Accordingly, as the upper limit frequency, the highest frequency is used among frequencies at which a necessary signal to noise ratio can be obtained at the shallowest position of the imaging depth. The upper limit frequency also differs depending on a measurement region or a disease, and is adjusted in accordance with the preference of a user in some cases. The signal is affected by the attenuation depending frequency as described above. Thus, a graph as shown in FIG. 7(b) in which the horizontal axis represents a frequency and deep, medium, and shallow regions are arranged for each propagation distance shows that as the region becomes deeper, the center frequency is shifted to the lower frequency side. On the other hand, the electric noise is normally white noise, and is accordingly less dependent on a frequency.

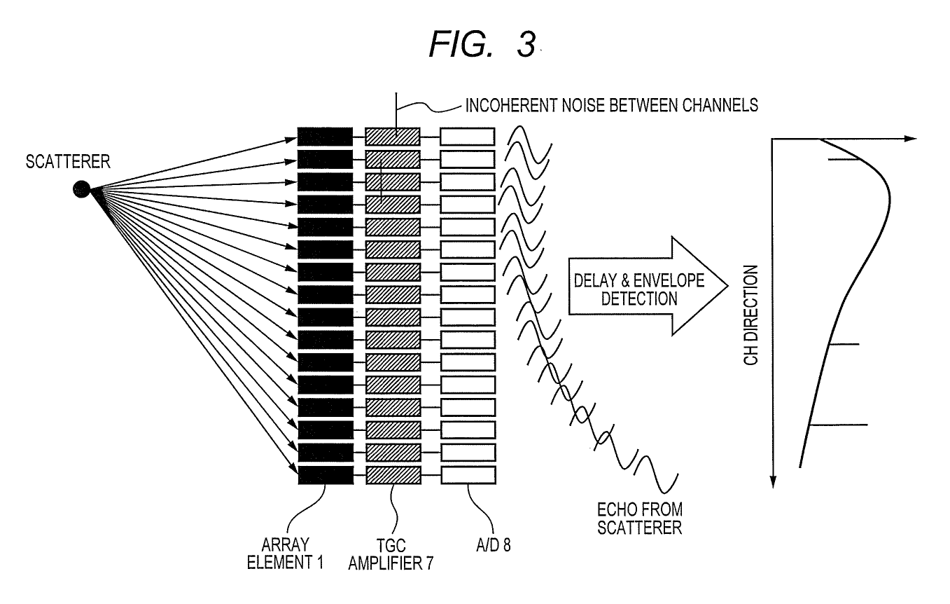

As shown in FIG. 3, the echo signals returned from a scatterer in a body are converted into electric signals at time differences in accordance with distances for the adjacent array elements. Specifically, the signals caused by the echo signals have constant continuity between channels. On the other hand, the electric noise mixed after being converted into an electric signal by the array element randomly enters between channels in the case of white noise. Thus, there is no continuity between channels. The present invention has been achieved by focusing on a difference in continuity in the ch direction between the signal and noise, and the signal and noise contained in the echo signals are separated from each other using continuity between channels to remove the noise. The signal strength distribution in the ch direction at a certain time is schematically shown in the rightmost part of FIG. 3. Here, the acoustic signal has continuity, and the electric noise appears as a signal without continuity. Accordingly, filtering is performed between channels before the delay and sum process, and the electric noise is separated and removed from the signal. In a process for an output of the beamformer as being performed in a normal diagnosing device, it is principally impossible to check a difference in continuity focused on by the present invention in the delay and sum process.

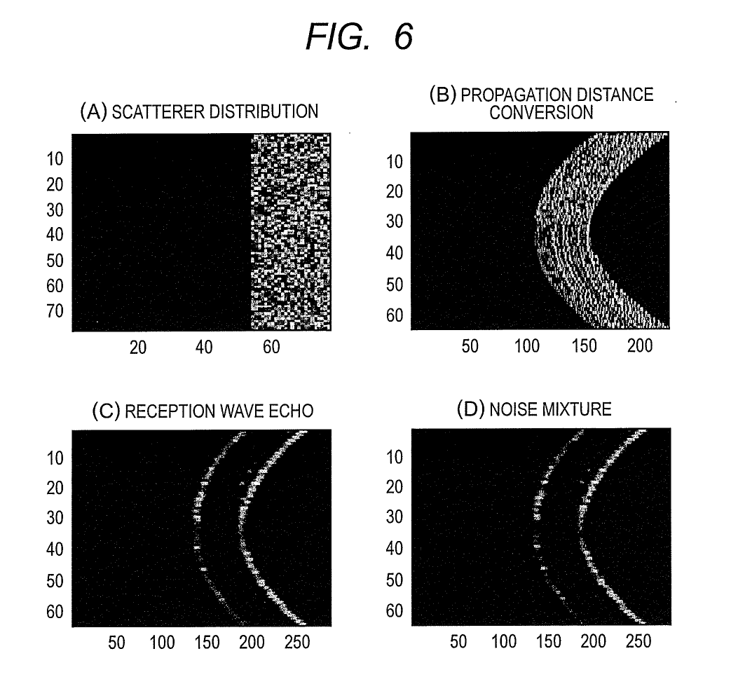

Next, effects of the present invention were quantitatively estimated by a real calculation simulation. The parameters used in the calculation were as follows: number of elements (number of channels), 64; center frequency, 7.5 MHz; pitches of elements, 0.2 mm; number of cycles of transmission waveforms, 2; focal distance, 50 mm; sampling time of simulation, 1/32 of cycle; acoustic velocity, 1540 m/s; intervals of scatterers: 1/16 of wavelength; and space where the scatterer was placed, 1×1 mm. Under the conditions, the echo signals from random scatterers were received by each element, and the transfer function and convolution of the array element were performed to be converted into waveforms on the temporal axis. In this case, the space of the scatterer was assumed to be in the focal area of the transmission beam. After being converted into waveforms on the temporal axis, electric noise was randomly added to perform a delay process between the elements, and the inter-channel filtering of the present invention was performed. After the filtering, an adding process between channels was performed. The scatterer existed in one half of the space of the scatterer and no scatterer existed in the other half thereof. Accordingly, the signal to noise ratio was evaluated. It should be noted that the simulation was performed so that an area without signals was a finite dynamic range in which the bit width of the analog to digital conversion was 16 bits. FIG. 6 show signal distribution in which FIG. 6(a) shows scatterer distribution in a real simulation, FIG. 6(b) shows correction of the scatterer positions in accordance with propagation distances, FIG. 6(c) shows reception wave echoes, and FIG. 6(d) shows a state after mixing noise. The signal distribution shown in FIG. 6(d) was filtered and the delay and sum process was performed to evaluate the signal to noise ratio.

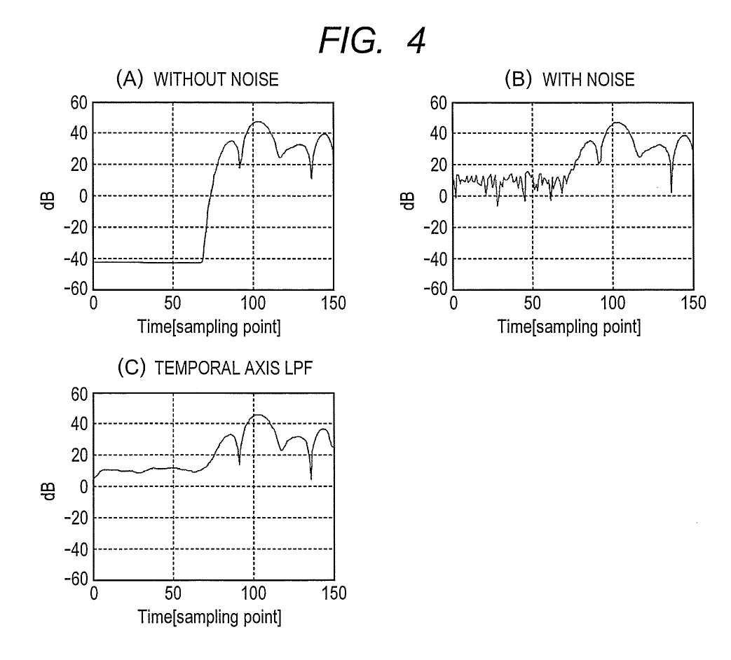

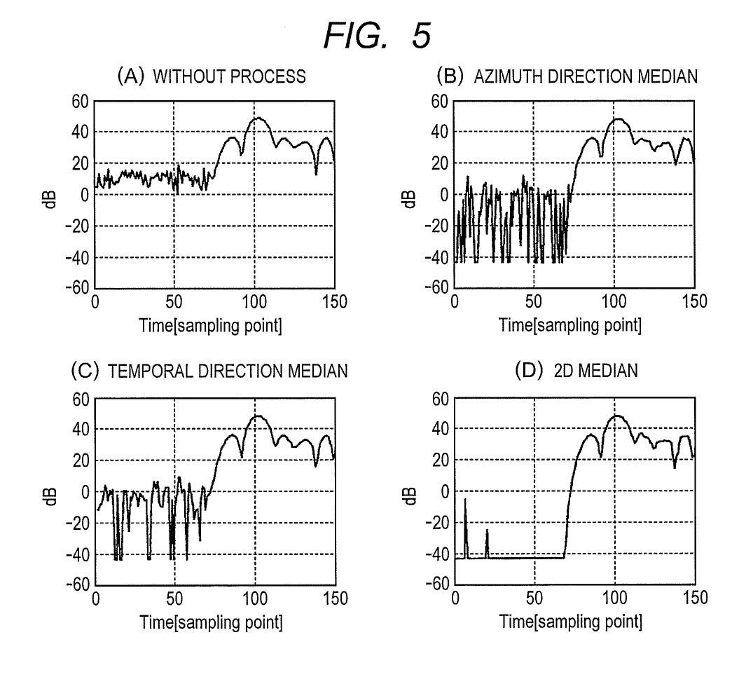

FIG. 4(a) shows echo data on the temporal axis after the delay and sum process in the case where no noise was mixed. No signals exist in an area where the number of sampling points is 60 or smaller, and signals exist in an area where the number of sampling points is 70 or larger. The average value of the noise strength was subtracted from the average value of the signal strength to evaluate the signal to noise ratio. FIG. 4(b) is a case with noise in which the signal to noise ratio was 28 dB. FIG. 4(c) shows a result obtained by a conventional method in which low-pass filters on the temporal axis were applied to each channel and the signal to noise ratio was 27 dB, which means little improvement. Next, FIG. 5 show results obtained by applying the present invention. FIG. 5(a) shows a case without filters same as FIG. 4(b). FIGS. 5(b) and 5(d) show results of the present invention, and FIG. 5(c) shows a result obtained by separating and removing noise in the temporal axis direction as a target. In any case, a 3×1 median filter was used to remove noise. The median filter is a filter to output a median value among input values. The median filter is effective in removing noise with a peculiar value relative to surrounding data. As a result of using the median filter between channels in FIG. 5(b), the signal to noise ratio was 56 dB. As a result of using the median filter on the temporal axis in FIG. 5(c), the signal to noise ratio was 44 dB. As a result of using the 2D median filter in FIG. 5(d), the signal to noise ratio was 82 dB. By comparing the improvement rates of the signal to noise ratios, the improvement rate in FIG. 5(b) of the present invention was 28 dB and that in FIG. 5(d) was 54 dB. On the other hand, the improvement rate using the median filter on the temporal axis was 16 dB. As described above, the maximum improvement rate of the signal to noise ratio according to the present invention was 54 dB, and the present invention is advantageous in suppressing noise with an improvement rate of 38 dB, namely, about one hundred times as compared to the method (c) analogical from the conventional method. If it is assumed that the center frequency is 10 MHz and the attenuation depending frequency of a living body is 0.6 dB/MHz/cm, 38 dB is equal to improvement of penetration by 6 cm.

As a filter between channels, the median filter has been described above as an example. The concept of the present invention is a method in which a signal without continuity is separated and removed through a signal with continuity between channels. In the viewpoint, a bandpass filter related to spatial frequencies can be used other than the median filter. The delay and sum process originally functions as a low-pass filter in the spatial method, and thus there is no difference.

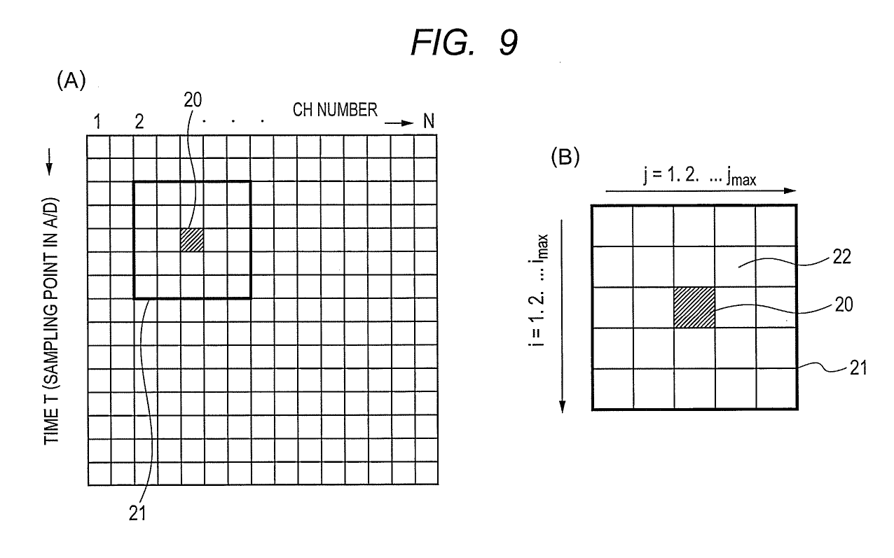

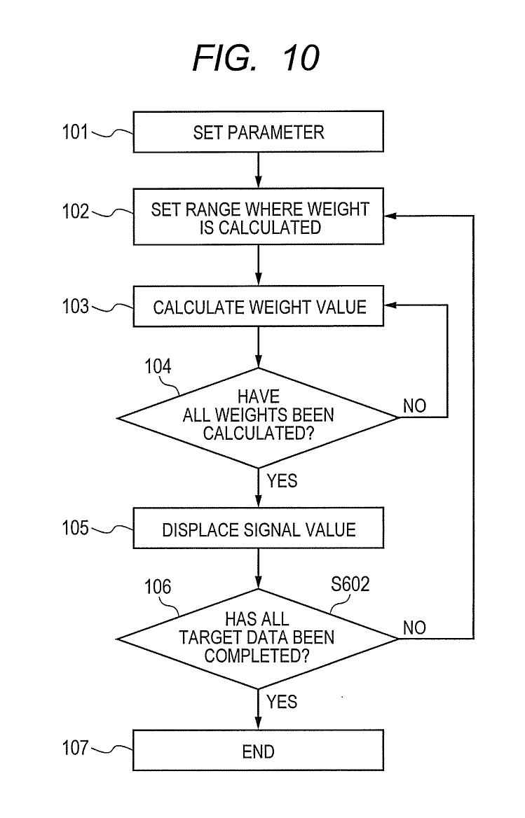

On the other hand, a filter that adaptively functions to the characteristics of a signal can be also used. For example, a weight-variable adaptive filter described below using each of FIG. 9 and FIG. 10 can be used. The strength of a point where the strength is calculated among data input into the adaptive filter is represented as and the size of a focused area (an area where the weight is calculated) for calculating an output of the strength is represented as iₘₐₓ×jₘₐₓ. The larger the size is, the more the filer is effective. However, the computation speed accordingly becomes slower. Hereinafter, the signal strength at coordinates (I and j) in the focused area is represented as Iᵢⱼ. The position of data where the calculation is performed, and data in a range determined by i and j are set, and a weight wᵢⱼ is calculated on the basis of a weighting function to be described later. When the weight calculation is performed for all points in the set range, a filter output Iₒ′ is obtained by Equation 1, and data to be calculated is shifted. If the calculation for the all points in the data is completed, the filter process is completed.

I₀′=Σwᵢⱼ×Iᵢⱼ/ΣIᵢⱼ Equation 1

Σ: adding in a range from i and j (each starting from 1) to iₘₐₓ and jₘₐₓ.

Next, the weighting function will be described. As a function by which a weight is monotonically decreased as the difference between I₀ and Iᵢⱼ becomes larger, the Gaussian function or an even-ordered polynomial equation can be used. The computing process of Equation 1 is performed using the weighting function, so that only the noise components can be suppressed without deteriorating the spatial resolution. An example of the weight-variable adaptive filter has been described above. However, if a morphological filter (a filter to calculate the maximum or minimum value with a shape weight added), a spike removal filter, or a ripple removal filter is used, the similar effects can be expected.

Further, in the case where the present invention is applied to the ultrasonic diagnosing device, it is effective to dynamically change the shape (the size of the median filter in the azimuth direction or the cutoff frequency of the spatial frequency in the azimuth direction) of the inter-channel filter in accordance with the depth of the echo signal source. Because an aperture width is normally constant except a close range in the ultrasonic diagnosing device. Thus, as the depth becomes deeper, a ratio of the focal distance to the aperture width becomes large and the beam width in the azimuth direction is widened. Further, the frequencies are cut from the high-frequency components due to the living body-dependent attenuation. Thus, echoes from a deeper position contain more low-frequency components, contributing to widening of the beam width. If it is assumed that each sampling point is in focus by the dynamic focus in consideration of reception wave beams, for example, a diffraction angle θ can be approximated by Equation 2 in the case of continuous waves in which the aperture width is W, the focal distance is L, the frequency is f, and the acoustic velocity of a living body is v.

θ=sin⁻¹(v/2fw) Equation 2

The beam width at the reception wave focal point is L×tan θ. Thus, the beam width is in proportion to L and is in inverse proportion to f under the conditions where θ is small (the aperture is sufficiently wide relative to the wavelength). As a result, the coherence distance between channels becomes longer at a deeper position, and the optimum filter size accordingly becomes large. If the change is employed, noise can be optimally removed at each depth.

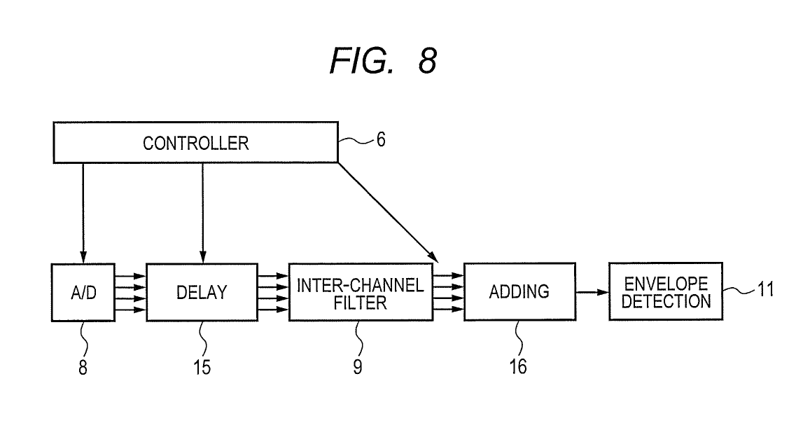

In the above study, selection of an acoustic signal and electric noise has been described. Using the method of the present invention, it is possible to discriminate a signal from the reception wave focal point from a signal (hereinafter, referred to as acoustic noise) from a position other than the reception wave focal point among the acoustic signals. In the reception wave beamforming, there are three kinds of signals as follows, as the acoustic signals from a point other than the reception wave focal point: (1) echo signals from a reflection source on a transmission and reception wave grating beam; (2) acoustic noise generated in such a manner that the echo signals from a point other than the reception wave focal point are received at the same timing as the arrival time of those from the focal point due to refraction or scattering during propagation; and (3) acoustic noise generated in such a manner that the echo signals from a point other than the reception wave focal point are received at the same timing as the arrival time of those from the focal point due to multiple reflection between the ultrasound probe and reflection objects in a living body. Among these, it is difficult to discriminate the signal (1) due to coherence with the signal from the reception wave focal point. On the other hand, for the signals (2) and (3), the distribution of reception times in each channel is different from that for signals from the reception wave focal point. When focusing on the difference between the characteristics, the acoustic noise caused by (2) and (3) can be reduced by applying the present invention. In the example of FIG. 1, the process is performed in the order of the analog to digital conversion 8, the inter-channel filter 9, and the receiving beamformer 10. However, the process of the receiving beamformer 10 is divided into a delay unit 15 and an adding unit 16 as shown in FIG. 8, and the process is performed in the order of the analog to digital conversion 8, the delay unit 15, the inter-channel filter 9, and the adding unit 16. In the delay curve, namely, the distribution of reception times in each channel for the acoustic noise is different from that for the signal. Thus, if the delay process in accordance with the signal is performed, the continuity of the acoustic noise between channels becomes small, and acoustic noise components can be suppressed by the inter-channel filter 9.

### Second Embodiment

In the first embodiment, there has been described an example of applying the method of removing electric noise to an ultrasonic cross-sectional image. In the embodiment, there will be described a case in which the present invention is applied to a Doppler blood flow measurement (a continuous wave Doppler measurement method and a pulse Doppler measurement method).

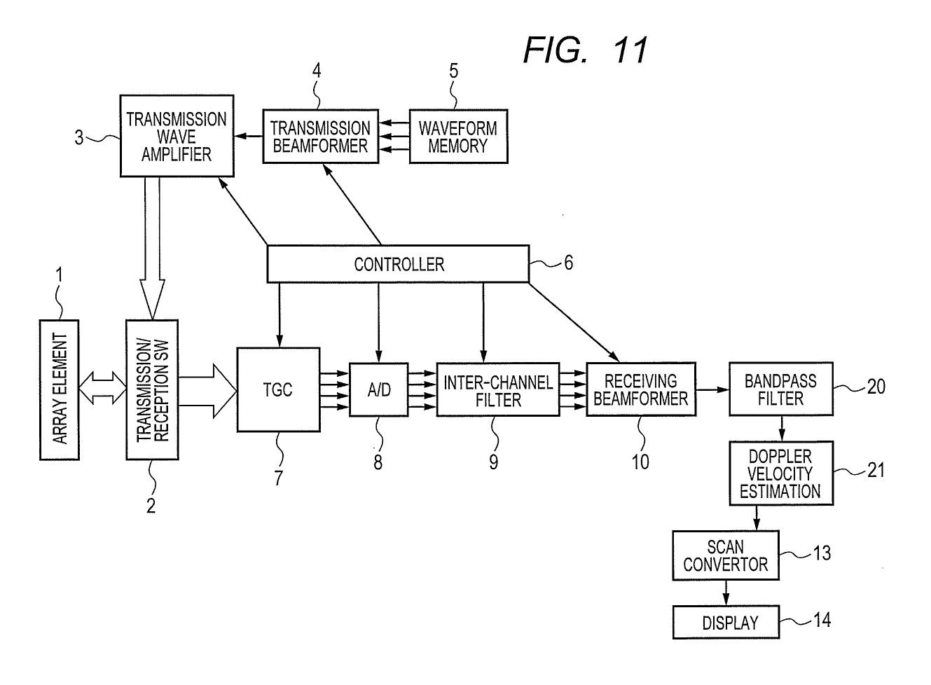

In the first place, there will be described a case in which the present invention is applied to the continuous wave Doppler measurement method. The continuous wave Doppler measurement method is a method in which while the azimuth direction where a reception wave beam is obtained is fixed, the frequency conversion of received data is performed by a method such as FFT and the speed of an echo source is estimated using effects of a Doppler shift by echo signals due to blood flow in accordance with the blood flow rate. As shown in FIG. 11, after the receiving beamformer process, the processes of the bandpass filter and estimation of the Doppler speed are performed to form an image of time changes of the blood flow rate. The processes performed between the analog to digital conversion and the receiving beamformer are the same.

Next, there will be described a case in which the present invention is applied to the pulse Doppler measurement method. Unlike the continuous wave Doppler measurement method, the frequency conversion is not performed in the temporal axis direction of echoes but is performed in a repetition data obtaining method in the pulse Doppler measurement method. In the case where the inter-channel filtering is performed only in the azimuth direction, the processes between the analog to digital conversion and the receiving beamformer are the same as described above.

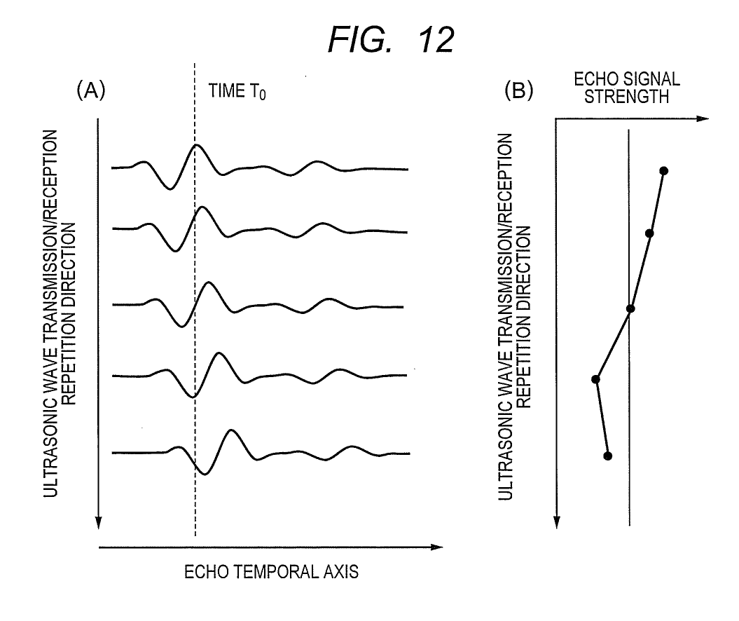

On the other hand, in the case where the analogy in which the two-dimensional median filter described in the first embodiment is applied in the channel direction and the temporal direction is applied to the pulse Doppler measurement method, coordinate axes are different from each other in the temporal direction. FIG. 12 show an example of the temporal axis of an echo signal, the temporal axis of ultrasonic wave transmission/reception repetition, and echo signals obtained by repeated transmission and reception. The repeated transmission/reception interval is adjusted in accordance with the movements of an object. Namely, the interval is optimized so that the speed can be estimated under the restriction of the Nyquist frequency. In order to adapt to a blood flow rate of 10 m/s to a few cm/s in a living body, a repetition frequency of about 100 Hz to 10 kHz is selected. In the case where the object does not move, the waveform of FIG. 12(a) do not vertically change at all. Thus, there are no changes of the signal of FIG. 12(b) corresponding to a specific sampling time T₀. However, in the case where the object moves in the depth direction while the signals are repeatedly obtained, the echo waveforms are changed as shown in FIG. 12(a), and phase rotation of the signals occurs in FIG. 12(b) corresponding to the specific sampling time T₀. The phase rotation speed is in proportion to the speed in the depth direction of the object.

The two-dimensional median filter in the ultrasonic wave transmission/reception repetition direction and the channel direction or the three-dimensional median filter in the ultrasonic wave transmission/reception repetition direction, the channel direction, and the temporal axis of echoes shown in FIG. 12 is applied in the present invention, and thus incoherent electric noise on the filter space can be selectively removed. The process after the incoherent noise is removed is the same as the normal pulse Doppler process.

### Third Embodiment

With the recent improvement in the performance of processors, an algorithm available as signal processing in an ultrasonic diagnosing device has been advanced. Especially, as a method of improving the resolution in the azimuth direction that has been regarded as a disadvantage in ultrasonic imaging from the past, the Capon method that has been developed in the fields of radar and mobile communications has been known. While a predetermined value has been used for a delay time irrespective of a reception signal in a conventional delay circuit, the Capon method is a method designed so that the delay time is optimized for each target reception data to realize the optimum resolution in the azimuth direction.

Specifically, the Capon method is a method of beamforming to optimize w so as to minimize the sum P=½ wRw^{t} of the product of given two channels calculated using a correlation matrix R=V^{t}V and a complex weight vector w to each channel while time-series data of each channel is represented as a vector V [v1 to vN] (N represents the number of channels and each v represents time-series data). The symbol with t in the upper right represents a transposed vector. When P is the smallest without constraint conditions, w=0. Thus, in order to make the beam output on the center axis other than 0, wa^{t} is set at 1 in the constraint conditions. In this case, “a” represents a mode vector that is composed of values obtained by adjusting differences between channel distances relative to the beam scanning direction to phase differences. “w” can be obtained by a variation method using w=R⁻¹a^{t}/(aR⁻¹a^{t}) at the time, the mode vector, the reciprocal of a correlation matrix. As preprocessing of the Capon beamforming, removing of incoherent noise of the present invention is advantageous. This is because the robustness of the Capon beamforming is improved.

### Fourth Embodiment

As a method of improving the signal to noise ratio, there is a coded excitation method. It is necessary to restrict the maximum value of ultrasonic strength in consideration of impacts on a living body. In order to increase energy to be transmitted under the restrictions, there has been used the coded excitation method spread in the field of radar in which a coded signal extended in the temporal axis direction is transmitted, a signal reflected in a target is received to be converted into an electric signal, and the electric signal is then compressed in the temporal axis direction by a filtering process to be restored to a pulse waveform.

In the case of the coded excitation method, a drive coded pulse extended in the temporal axis direction is used. When an ultrasound probe is driven with the drive coded pulse, an ultrasonic signal with a coded waveform is transmitted from the ultrasound probe to the inside of a living body, and is returned after being reflected by a reflector in the living body. If the ultrasonic signal is converted into an electric signal again using the ultrasound probe, a coded reception waveform can be obtained. The coded waveform is shortened on the temporal axis by the extended length of the drive signal using a decoding filter adapted to the drive coded pulse. As a result, a decoded waveform with large signal strength can be obtained with the same distance resolution as the case of pulse transmission/reception. As described above, transmission wave energy can be increased without enlarging the amplitude in the living body. It is effective to combine the method and the inter-channel filter process of the present invention. Making the coded waveform as long as possible has a great effect on the signal to noise ratio in the coded excitation method. However, if it becomes extremely long, the length of the waveform exceeds the reception wave dynamic focus area, resulting in failure in decoding the codes. Therefore, it is desirable to decode the codes prior to the delay and sum process for the reception wave so as not to be affected by the reception wave dynamic focus. In the case where the present invention is applied to the coded excitation method, the position of the inter-channel filter inserted is important. This is because a difference in coherence between channels occurs even in the state of the coded waveform. Accordingly, the order of the analog to digital conversion of reception, the inter-channel filter, decoding the codes, and the delay and sum process is only a solution in the present invention.

The present invention has been described above using typical examples. However, it is obvious that the present invention can be realized within a range where the technological concept of the present invention is not changed even if the element technique is changed.

### REFERENCE SIGNS LIST

1 . . . array element, 2 . . . transmission/reception switch, 3 . . . transmission wave amplifier, 4 . . . transmission beamformer, 5 . . . waveform memory, 6 . . . controller, 7 . . . time gain control amplifier, 8 . . . analog to digital conversion element, 9 . . . inter-channel filter, 10 . . . receiving beamformer, 11 . . . envelope detection, 12 . . . bandpass filter, 13 . . . scan convertor, 14 . . . display, 15 . . . delay unit, 16 . . . adding unit

1. An ultrasonic imaging device comprising:
an array element;
a transmitter that transmits ultrasonic waves to a target through the array element;
a receiver that delays and sums a reception wave signal received by the array element from the target;
a display that displays a cross-sectional image of the target from the reception wave signal output from the receiver; and
a controller that controls the transmitter and the receiver, wherein
the receiver includes an inter-channel filter that selectively suppresses electric noise, a receiving beamformer that selectively receives a beam, and an envelope detection unit, and the inter-channel filter performs a filtering process in which a signal and noise contained in an echo signal from the target are separated from each other using continuity between channels to remove the noise.

2. The ultrasonic imaging device according to claim 1, wherein
a process of the inter-channel filter is performed after analog to digital conversion and before an adding process between channels.

3. The ultrasonic imaging device according to claim 1, wherein
the inter-channel filter is a median filter.

4. The ultrasonic imaging device according to claim 1, wherein
the inter-channel filter is a two-dimensional median filter between channels and in a temporal axis.

5. The ultrasonic imaging device according to claim 1, wherein:
the inter-channel filter includes means that sets a data point range around each point of the echo data, and means that obtains a function for determining a weighting function based on a difference between the signal strength of each data and the strength of each data point in the data point range; the function has the maximum point when the difference is 0; the value of integral of the absolute value of the function in a range between negative infinity and positive infinity is finite; the weighting function for each data in the data point range is determined based on differential of the function; and the value obtained by adding the sum of products between the weighting function and the strength of each data in the data point range to the strength of the data is used as the signal strength of the result of the filtering process.

6. The ultrasonic imaging device according to claim 1, wherein
the filter size of the inter-channel filter is changed in accordance with the reception time of the echo data.

7. The ultrasonic imaging device according to claim 1, wherein
the noise separated and removed by the inter-channel filter is acoustic noise, together with electric noise, from a point other than a focal point and a sound source around the focal point.

8. The ultrasonic imaging device according to claim 1, wherein
a process of the inter-channel filter is performed after the analog to digital conversion and the delay process and before the adding process between channels.

9. The ultrasonic imaging device according to claim 1, wherein
a Doppler blood flow rate is particularly measured, and the inter-channel filter is a two-dimensional or higher filter among three dimensions of an ultrasonic transmission/reception repetition temporal axis, an axis on which channels are arranged, and a temporal axis of echo data.

10. The ultrasonic imaging device according to claim 2, wherein
delay data used in the adding process is calculated based on the Capon method.

11. The ultrasonic imaging device according to claim 2 wherein:
a waveform coded circuit to extend a transmission wave pulse on a temporal axis is provided; a composite circuit in which a transmission waveform is coded and transmitted, the transmission waveform is received, and then the pulse of the reception waveform is compressed is provided; and a process of the inter-channel filter is performed after the analog to digital conversion and before the adding process between channels.
